(19) United States
(12) Patent Application Publication
(10) Pub. No.: US 2007/0178123 A1
(43) Pub. Date: Aug. 2, 2007

(54) FLAVOR-ENHANCING COMPOSITIONS, METHOD OF MANUFACTURE, AND METHODS OF USE

(76) Inventors: Deborah Levenson, Morristown, NJ (US); Paula Elejalde, Randolph, NJ (US)

Correspondence Address: CANTOR COLBURN, LLP, 55 GRIFFIN ROAD SOUTH, BLOOMFIELD, CT, 06002, US

(21) Appl. No.: 11/627,621

(22) Filed: Jan. 26, 2007

Related U.S. Application Data
(60) Provisional application No. 60/762,677, filed on Jan. 27, 2006.

(51) Int. Cl. A61K 9/00; A61K 36/00

(52) U.S. Cl. 424/400; 424/725

(57) ABSTRACT

A flavor-enhancing composition for an ingestible product includes a medicament for the treatment of a cough, or a cold or flu symptom; a physiological cooling agent; and a high intensity sweetener. An undesirable flavor associated with the medicament, such as bitterness, is reduced when it is combined with the physiological cooling agent and the high intensity sweetener.

### CROSS REFERENCE TO RELATED APPLICATIONS

This application claims the benefit of U.S. Provisional Application Ser. No. 60/762,677, filed Jan. 27, 2006. This provisional application is incorporated herein by reference.

### FIELD

This disclosure is related to flavor-enhancing compositions and comestibles containing such compositions, as well as methods for the manufacture and use thereof. In particular, this disclosure relates to compositions that impart a bitter or unpleasant off-note taste-masking effect for medicaments when orally consumed by an individual.

### BACKGROUND

Comestible products containing medicaments such as dextromethorphan may have a bitter or unpleasant off-note taste that adversely affects the overall flavor of the product. In many instances, the flavor of comestible products containing medicaments would be improved by diminishing or removing the bitter or unpleasant off-note tastes, while at the same time preserving or enhancing the contribution made to the overall flavor by the non-bitter flavor components.

Previous efforts to mask bitter or unpleasant off-note tastes have included the use of intense sweeteners. Because each intense sweetener is chemically distinct, each sweetener presents a different challenge with respect to the actual use of such sweetener in a comestible product. For example, some intense sweeteners present stability problems, such as aspartame, which exhibits instability in the presence of aldehydes, ketones, moisture, and the like. Other intense sweeteners have an associated bitter or off-note taste, such as saccharin, steviosides, acesulfame K, glycyrrhizin, dipotassium glycyrrhizin, glycyrrhizic acid ammonium salt, and thaumatin. Because of the chemical distinctness of each sweetener or flavoring agent, it is often challenging to choose the appropriate combination of sweetener or flavoring agent to mask the bitter or unpleasant off-note taste of flavoring agents. Additional efforts to mask bitter or unpleasant off-note tastes have included the preparation of modified release formulations. Modified release of bitter or unpleasant off-note tastes may be obtained by encapsulation, partial encapsulation, or partial coating, entrapment or absorption with high or low water soluble materials or water insoluble materials.

Despite the foregoing efforts, there nonetheless remains a need in the art for flavor-enhancing compositions that mask the bitter or unpleasant off-note taste of medicaments such as dextromethorphan in comestible products.

### SUMMARY

One embodiment is a flavor-enhancing composition for a comestible product comprising a medicament for the treatment of a cough or a cold or flu symptom. a physiological cooling agent, and a high intensity sweetener.

One embodiment is a flavor-enhancing composition for a comestible product, comprising: about 1 to about 50 weight percent sucralose; about 0.01 to about 15 weight percent neotame; about 0.1 to about 50 weight percent sodium citrate; about 1 to about 50 weight percent acidulant; about 1 to about 90 weight percent menthol; about 0.01 to about 20 weight percent N-ethyl-p-menthane-3-carboxamide, menthyl glutarate, or a combination thereof; and about 0.01 to about 10 weight percent dextromethorphan; wherein all weight percents are based on the total weight of the flavor-enhancing composition.

One embodiment is a method for the manufacture of a flavor-enhancing composition for a comestible product, comprising combining a medicament for the treatment of a cough or a cold or flu symptom, a physiological cooling agent, and a high intensity sweetener.

One embodiment is a flavor-enhanced comestible product comprising: a comestible composition, a medicament for the treatment of a cough or a cold or flu symptom, a physiological cooling agent and a high intensity sweetener.

One embodiment is a method for the manufacture of a comestible, comprising: combining a comestible composition, a medicament for the treatment of a cough or a cold or flu symptom, a physiological cooling agent, and a high intensity sweetener.

One embodiment is a method for enhancing the flavor of a comestible product comprising: providing to a consumer a comestible product comprising a comestible composition, a medicament for the treatment of a cough or a cold or flu symptom, a physiological cooling agent, and a high intensity sweetener; and instructing the consumer to apply the comestible product to the oral cavity of an individual and allow the comestible to dissolve.

One embodiment is a method for enhancing the flavor of a comestible product comprising: adding to a comestible composition a medicament for the treatment of a cough or a cold or flu symptom, a physiological cooling agent, and a high intensity sweetener.

One embodiment is a method for enhancing the flavor of a comestible product comprising: applying to the oral cavity of an individual a comestible product comprising a comestible composition, a medicament for the treatment of a cough or a cold or flu symptom, a physiological cooling agent, and a high intensity sweetener; and allowing the comestible to release the above-described flavor-enhancing composition from the comestible into the oral cavity, thereby enhancing the flavor of the comestible product.

One embodiment is a method for the treatment of a cough, or a cold or flu symptom, in a subject in need of such treatment, the method comprising: administering to the subject a flavor-enhanced comestible product comprising a comestible composition, a medicament for the treatment of a cough or a cold or flu symptom, a physiological cooling agent, and a high intensity sweetener.

These and other embodiments are described in detail below.

### DETAILED DESCRIPTION

It has been found by the inventors hereof that a bitter or off-note taste of a medicament for the treatment of a cough, or a cold or flu symptom, can be decreased or nullified by a flavor-enhancing composition comprising a combination of the medicament with a physiological cooling agent, for example menthol, and a high intensity sweetener, for example neotame or sucralose or both. This result is particularly surprising because many physiological cooling agents and high-intensity sweeteners are themselves known to have a bitter or off-note taste. In some embodiments, a bitter or unpleasant off-note taste is imparted by the medicament or the high intensity sweetener or the physiological cooling agent or a combination of two of the foregoing, and a bitter or unpleasant off-note taste of a combination of the medicament, the high intensity sweetener, and the physiological cooling agent is less than the bitter or unpleasant off-note taste imparted by the medicament or the high intensity sweetener or the physiological cooling agent or the combination of two of the foregoing. In other words, in some embodiments there is a synergistic reduction of a bitter or off-note taste. The compositions are of particular utility in comestibles such as lozenges and gums.

A flavor-enhancing composition for a comestible product accordingly comprises a medicament for the treatment of cough or a cold symptom or a flu symptom, a physiological cooling agent, and a high intensity sweetener. The flavor-enhancing composition masks any bitter or off-note tastes associated with the medicament. As a result, improved compliance with dosing regimens occurs for patients in need of treatment for coughs, or a cold or flu symptom.

Medicaments for the treatment of a cough, or a cold or flu symptom include elements, compounds or materials, alone or in combination, that have been used for, or have been shown to be useful for, the amelioration of at least one symptom commonly associated with cough, colds, or influenza. It is to be understood that a “medicament for the treatment of a cough, or a cold or flu symptom” includes medicaments that are also useful for the treatment of cold-like or flu-like symptoms arising from other sources, such as allergies, adverse environmental conditions, and the like. Cold symptoms, cold-like symptoms, flu symptoms, and flu-like symptoms as used herein include cough, coryza, nasal congestion, upper respiratory infections, allergic rhinitis, otitis, sinusitis, sneezing, and the discomfort, pain, fever and general malaise associated with colds, flu, allergies, adverse environmental conditions, and the like.

Examples of general categories of medicaments for the treatment of a cough, or a cold or flu symptom include antihistamines, decongestants (sympathomimetics), antitussives (cough suppressants), anti-inflammatories, homeopathic agents, expectorants, anesthetics, demulcents, analgesics, anticholinergics, throat-soothing agents, antibacterial agents, and antiviral agents. Some of these medicaments may serve more than one purpose. The pharmaceutically acceptable salts and prodrugs of the medicaments are also included unless specified otherwise. Two or more medicaments that have activity against the same or different symptoms of colds or coughs can be used together in a combination.

Exemplary antihistamines include azatadine, bromodiphenhydramine, brompheniramine, brompheniramine maleate, carbinoxamine, carbinoxamine maleate, cimetidine, chlolpheniramine, chlorpheniramine maleate, dexchlorpheniramine, diphenhydramine, diphenhydramine hydrochloride, doxylamine, phenindamine, pheniramine, phenyltoloxamine, pyrilamine, promethazine, triprolidine, loratadine, ranitidine, chlorcyclizine, terfenadine, clemastine fumarate, dimenhydrinate, prilamine maleate, tripelennamine hydrochloride, tripelennamine citrate, hydroxyzine pamoate, hydroxyzine hydrochloride, cyclizine lactate, cyclizine hydrochloride, meclizine hydrochloride, acrivastine, cetirizine hydrochloride, astemizole, levocabastine hydrochloride, and cetirzine.

Exemplary decongestants include agents such as levopropoxyphene napsylate, noscapine, carbetapentane, caramiphen, chlophedianol, pseudoephedrine hydrochloride, phenylephrine, phenylpropanolamine, diphenhydramine, glaucine, pholcodine, benzonatate, ephedrine, epinephrine, levodesoxyephedrine, oxymetazoline, naphazoline, propylhexedrine, and xylometazoline.

Antitussives help relieve coughing. Examples of antitussives include such as codeine, dihydrocodeine, hydrocodone and hydromorphone, carbetapentane, caramiphen, hydrocodone bitartrate, chlorphedianol, noscarpine, and dextromethorphan.

Exemplary expectorants include guaifenesin, aniseed, blood root, coltsfoot, elderflower, golden seal, grindelia, hyssop, lungwort, mullein, senega, thuja, thyme, veivain, glyceryl guaiacolate, terpin hydrate, N-acetylesteine, bromhexine, ambroxol, domiodol, 3-iodo-1,2-propanediol and wild cherry, ammonium chloride, calcium iodide, iodinated glycerol, potassium guaiacolsulfonate, potassium iodide, and sodium citrate.

Exemplary anaesthetics include etomidate, ketamine, propofol, and benodiazapines (e.g., chlordiazepoxide, diazepam, clorezepate, halazepam, flurazepam, quazepam, estazolam, triazolam, alprozolm, midazolam, temazepam, oxazepam, lorazepam), benzocaine, dyclonine, bupivacaine, etidocaine, lidocaine, mepivacaine, promoxine, prilocalne, procaine, proparcaine, ropivacaine, tetracaine. Other useful agents may include amobartital, aprobarbital, butabarbital, butalbital mephobarbital, methohexital, pentobarbital, phenobarbital, secobarbital, thiopental, paral, chloral hydrate, ethchlorvynol, clutethimide, methprylon, ethinamate, and meprobamate.

Exemplary analgesics include opioids such as morphine, mepidine, dentanyl, sufentranil, alfentanil, aspirin, salicylamide, sodium salicylate, acetaminophen, ibuprofen, indomethacine, naproxen, atrin, isocome, midrin, axotal, firinal, phrenilin, ergot and ergot derivatives (wigraine, cafergot, ergostat, ergomar, dihydroergotamine), and imitrex.

Exemplary anticholinergics include homatropine, atropine, scopolamine HBr, L-hyoscyamine, L-alkaloids of belladonna, tincture of belladonna alkaloids, homatropine HBr, homatropine methylbromide, methscopolamine, anisotropine, anisotropine with phenobarbital, clindinium, glycopyrrolate, hexocyclim, isopropamide, mepenzolate, methantheline, oxyphencyclimine, propantheline, tridihexethyl, dicyclomine, scopolamine, atropine, dicyclomine, flavoxate, ipratropium, oxybutynin, pirenzepine, tiotropium, tolterodine, tropicamide, trimethaphan, atracurium, doxacurium, mivacurium, pancuronium, tubocurarine, vecuronium, and suxamethonium chloride.

Exemplary demulcents include coltsfoot, comfrey, pectin, glycerogelatin, mucilages, Icelandic moss, Irish moss, linseed, locust bean, slippery elm bark, quince seed, corn silk, couchgrass, flaxseed, lungwort, liquorice, mallow, marshmallow, mullein, oatmeal, parsley piert, and slippery elm.

Exemplary antibacterial agents include those within the antibiotic classes of aminoglycosides, cephalosporins, macrolides, penicillins, quinolones, sulfonamides, and tetracyclines. Specific exemplary antibiotic agents include naficillin, oxacillin, vancomycin, clindamycin, erythromycin, trimethoprimsulphamethoxazole, rifampin, ciprofloxacin, broad spectrum penicillin, amoxicillin, gentamicin, ceftriazoxone, cefotaxime, chloramphenicol, clavunate, sulbactam, probenecid, doxycycline, spectinomycin, cefixime, penicillin G, minocycline, β-lactamase inhibitors; meziocillin, piperacillin, aztreonam, norfloxacin, trimethoprim, ceftazidime, dapsone, neomycin, azithromycin, clarithromycin, amoxicillin, ciprofloxacin, and vancomycin.

Antiviral agents specifically or generally modulate the biological activity of viruses such as picornavirus, influenza virus, herpes viruses, herpes simplex, herpes zoster, enteroviruses, varicella and rhinovirus, which are associated with the common cold. Exemplary antiviral agents include acyclovir, trifluridine, idoxorudine, foscarnet, ganciclovir, zidovudine, dideoxycytosine, dideoxyinosine, dipyridamole, stavudine, cidofovir, famciclovir, valaciclovir, valganciclovir, acyclovir, didanosine, zalcitabine, rifimantadine, saquinavir, indinavir, ritonavir, ribavarin, nelfinavir, adefovir, nevirapine, delavirdine, efavirenz, abacavir, amantadine, emtricitabine, entecavir, tenofovir, zanamivir, oseltamivir, ICI 130,685, impulsin, pleconaril, penciclovir, vidarabine, and cytokines.

Exemplary anti-inflammatories include salicylic acid derivatives (e.g., aspirin), paraminophenol derivatives (e.g. acetaminophen), indole and indene acetic acids (indomethacin, sulindac and etodalac), heteroaryl acetic acids (tolmetin diclofenac and ketorolac), aryl propionic acid derivatives (ibuprofen, naproxen, ketoprofen, fenopren, ketorlac, carprofen, oxaprozine), anthranilic acids (mefenamic acid, meclofenamic acid), and enolic acids (piroxicam, tenoxicam, phenylbutazone and oxyphenthatrazone).

In one embodiment the medicament for the treatment of cough, or cold or flu symptoms is an antihistamine, a decongestant, an antitussive, an antiinflammatory, a homeopathic agent, an expectorant, a demulcent, an analgesic, a throat-soothing agent, or a combination of at least two of the foregoing medicaments. In a specific embodiment, the medicament is a decongestant, an antitussive, an anti-inflammatory, an expectorant, a demulcent, an analgesic, a throat-soothing agent, or a combination of at least two of the foregoing medicaments. In another specific embodiment the medicament is a decongestant, an antitussive, an expectorant, a demulcent, a throat-soothing agent, or a combination of at least two of the foregoing medicaments. In another specific embodiment, the medicament is an antitussive, an expectorant, a demulcent, a throat-soothing agent, or a combination of at least two of the foregoing medicaments.

In still another specific embodiment, the medicament for the treatment of cough, cold or flu symptoms is an antitussive, for example dextromethorphan. Dextromethorphan is also known as racemethorphan and as 3-methoxy-17-methyl-9(alpha), 13 (alpha), 14(alpha)-morphinan hydrobromide monohydrate. In some embodiments, dextromethorphan can be combined with caffeine, aspirin, acetaminophen, ibuprofen, dyclonine, chlorpheniramine maleate, pseudoephedrine hydrochloride, benzocaine, or naproxen.

The amount of medicament or its acid addition salt used in the comestible product varies depending upon the therapeutic dosage recommended or permitted. In general, the amount of medicament present is the ordinary dosage used in the treatment of cough, or cold or flu symptoms. Such dosages are known to the skilled practitioner.

Physiological cooling agents are additives that provide a cooling or refreshing effect in the mouth, in the nasal cavity, or on skin. Physiological cooling agents include polyols exhibiting a negative heat of solution, including xylitol, erythritol, dextrose, and sorbitol, and combinations of at least two of the foregoing; menthyl-group containing cooling agents such as p-menthane, menthone, menthone ketals including menthone glycerol ketals, menthyl alcohols including menthol (2-isopropyl-5-methylcyclohexanol), L-menthol and its natural and synthetic derivatives, (−)-(1R,3R,4S)-3-p-menthanol, (−)-(1R,3R,4S)-8-p-menthen-3-ol, menthane diols including p-menthane-2,3-diol and p-menthane-3,8-diol, menthol glyceryl ether, menthoxyalkane alcohols, 6-isopropyl-9-methyl-1,4-dioxaspiro[4,5]decane-2-methanol, 3-(1-menthoxy)ethan-1-ol, 3-(1-menthoxy)propan-1-ol, 3-(1-menthoxy)butan-1-ol, menthoxyalkane diols, 3-(1-menthoxy)propane-1,2-diol (e.g., from Takasago, FEMA 3784), 3-(1-menthoxy)-2-methylpropane-1,2-diol, WS-30, p-menthane-3-carboxylic acid glycerol ester, menthol methyl ether, menthyl esters of aliphatic and aromatic monocarboxylic acids, menthyl acetate, menthyl lactate (e.g., from Haarman & Reimer, FEMA 3748, tradename FRESCOLAT® type ML), menthyl 3-hydroxybutyrate, menthyl 4-hydroxypentanoate, menthyl salicylate, menthyl pyrrolidone carboxylate (trade name QUESTICE), monomenthyl esters of aliphatic dicarboxylic acids, monomenthyl glutarate, monomenthyl succinate, alkali metal salts and alkaline earth metal salts of the foregoing, hydroxymethyl and hydroxyethyl derivatives of p-menthane, p-menthane carboxamides, N-aryl menthane carboxamides, N-ethyl-p-menthane-3-carboxamide (WS-3), 1-menthylacetic acid N-ethylamide, N,N-dimethyl menthyl succinamide, N-tert-butyl-p-menthane-3-carboxamide (WS-14), ethyl 3-(p-menthane-3-carboxamido)acetate (also known as WS-5; ethyl ester of N-[[5-methyl-2-(1-methylethyl)cyclohexyl]carbonyl]glycine, CAS Reg. No. 39668-74-1), menthol glycol carbonates, menthol ethyleneglycol carbonate, menthol propyleneglycol carbonate; substituted cyclohexane alcohols, trimethylcyclohexanol, isopulegol; cyclohexane carboxamides, N-methyl-2-isopropyl-bicyclo(2.2.1)heptane-2-carboxamide; acyclic carboxamides, N,2,3-trimethyl-2-isopropyl butanamide (WS-23), N-ethyl-trans-2-cis-6-nonadienamide; 1-methyl-cyclohexanecarboxylic acid (3-methoxy-phenyl)-amide, 1-methyl-cyclohexanecarboxylic acid (4-cyano-phenyl)-amide, 2-methyl-bicyclo[2.2.1]hept-5-ene-2-carboxylic acid (4-cyano-pheny)-amide, 2-methyl-bicyclo[2.2.1]hept-5-ene-2-carboxylic acid (4-methoxy-phenyl)-amide, 3-isopropyl-1-methyl-cyclopentanecarboxylic acid (4-methoxy-phenyl)-amide, 3-isopropyl-1-methyl-cyclopentanecarboxylic acid (3-cyano-phenyl)-amide, adamantane-1-carboxylic acid (4-methoxy-phenyl)-amide, 2-tert-butyl-cyclopentanecarboxylic acid (4-methoxy-phenyl)-amide, 2-tert-butyl-cyclohexanecarboxylic acid (2-methoxy-phenyl)-amide, 2-tert-butyl-cyclopentanecarboxylic acid (4-hydroxymethyl-phenyl)-amide, 2-tert-butyl-cyclopentanecarboxylic acid (4-acetyl-phenyl)-amide, 2-tert-butyl-cyclopentanecarboxylic acid (4-cyano-pheriyl)-amide, 2-tert-butyl-cyclohexanecarboxylic acid (4-hydroxymethyl-phenyl)-amide, 2-tert-butyl-cyclohexanecarboxylic acid (4-acetyl-phenyl)-amide, and 2-tert-butyl-cyclohexane-carboxylic acid (4-cyano-phenyl)-amide; thienopyrimidine cooling agents; substituted ureas and sulfonamides; 2-mercapto-cyclodecanone; hydroxycarboxylic acids with 2-6 carbon atoms; plant extracts including Japanese mint oil, peppermint oil, and eucalyptus extract; substituted derivatives of the foregoing; and combinations of at least two of the foregoing. These and other suitable cooling agents are further described in U.S. Pat. Nos. 4,136,163 and 4,150,052 and 4,178,459 and 4,190,643 and 4,193,936 and 4,226,988 to Watson et al., U.S. Pat. Nos. 4,230,688 and 4,032,661 and 4,153,679 and 4,296,255 to Rowsell et al., U.S. Pat. No. 4,459,425 to Amano et al., U.S. Pat. No. 5,009,893 to Cheruki et al., U.S. Pat. No. 5,266,592 to Grub et al., U.S. Pat. No. 5,698,181 to Luo, U.S. Pat. Nos. 5,725,865 and 5,843,466 to Mane et al., U.S. Pat. No. 6,231,900 to Hanke, U.S. Pat. No. 6,277,385 to Luke, U.S. Pat. Nos. 6,280,762 and 6,306,429 and 6,432,441 to Bealin Kelly et al., U.S. Pat. Nos. 6,455,080 and 6,627,233 and 7,078,066 to Wolf et al., U.S. Pat. No. 6,783,783 to Clark et al., U.S. Pat. No. 6,884,906 to Dewis et al., U.S. Pat. No. 7,030,273 to Sun, and U.S. Pat. No. 7,090,832 to Zanone et al.; U.S. patent application Publication Nos. U.S. 2004/0175489 of Clark et al., U.S. 2004/0191402 of Stawski et al., U.S. 2005/0019445 of Wolf et al., U.S. 2005/0222256 and U.S. 2005/0265930 of Erman et al., U.S. 2006/0159819 of Witkewitz et al., and U.S. 2006/0249167 of Giersch et al.; European Patent Application No. EP 1689256 A1 of Shimizu et al.; and International Patent Application Nos. WO 2005/082154 A1 of Johnson et al., WO 2005/099473 A1 of Vanrietvelde et al., WO 2006/058600 A1 of Foster et al., WO 2006/092076 A2 of Galopin et al., and WO 2006/125334 A1 of Bell et al. In some embodiments, the composition excludes one or more of the foregoing cooling agents.

In some embodiments, the physiological cooling agent is a menthyl-based coolant. A menthyl-based coolant is a physiological cooling agent comprising a methyl group. Menthyl-based coolants include menthol and menthol derivatives. Menthol (also known as 2-(2-propyl)-5-methyl-1-cyclohexanol) is available in artificial form, or naturally from sources such as peppermint oil. Menthol derivatives included menthyl ester-based and menthyl carboxamide-based cooling compounds such as menthyl carboxamide, N-ethyl-p-menthane carboxamide, monomenthyl succinate, monomenthyl-alpha, monomenthyl methyl succinate, monomenthyl glutarate, menthyl 2-pyrrolidone-5-carboxylate, monomenthyl 3-methyl maleate, menthyl acetate, menthyl lactate, menthyl salicylate, 2-isopropanyl-5-methylcyclohexanol, 3,1-menthoxypropane 1,2-diol, menthane, menthone, menthone ketals, menthone glycerol ketals, menthyl glutarate esters, or a combination of at least two of the foregoing. A specific exemplary coolant is N-ethyl-p-menthane-3-carboxamide, commercially available as WS-3.

A “high intensity sweetener” as used herein means agents having a sweetness at least 100 times that of sugar (sucrose) on a per weight basis, specifically at least 500 times that of sugar on a per weight basis. In one embodiment the high intensity sweetener is at least 1,000 times that of sugar on a per weight basis, more specifically at least 5,000 times that of sugar on a per weight basis. The high intensity sweetener can be selected from a wide range of materials, including water-soluble sweeteners, water-soluble artificial sweeteners, water-soluble sweeteners derived from naturally occurring water-soluble sweeteners, dipeptide based sweeteners, and protein based sweeteners. Combinations comprising one or more sweeteners or one or more of the foregoing types of sweeteners can be used. Without being limited to particular sweeteners, representative categories and examples include:

(a) water-soluble sweetening agents such as monellin, steviosides, lo han quo, glycyrrhizin, dihydroflavenol, monatin, and L-aminodicarboxylic acid aminoalkenoic acid ester amides, such as those disclosed in U.S. Pat. No. 4,619,834, or a combination of at least two of the foregoing;

(b) water-soluble artificial sweeteners such as soluble saccharin salts, e.g., sodium or calcium saccharin salts, cyclamate salts, acesulfame salts, such as the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (Acesulfame-K), the free acid form of saccharin, or a combination of at least two of the foregoing;

(c) dipeptide based sweeteners, for example the L-aspartic acid derived sweeteners such as L-aspartyl-L-phenylalanine methyl ester (Aspartame), N—[N-(3,3-dimethylbutyl)-L-α-aspartyl]-L-phenylalanine 1-methyl ester (Neotame), and materials described in U.S. Pat. No. 3,492,131, L-alpha-aspartyl-N-(2,2,4,4-tetramethyl-3-thietanyl)-D-alaninamide hydrate (Alitame), methyl esters of L-aspartyl-L-phenylglycerine and L-aspartyl-L-2,5-dihydrophenyl-glycine, L-aspartyl-2,5-dihydro-L-phenylalanine; L-aspartyl-L-(1-cyclohexen)-alanine, or a combination of at least two of the foregoing;

(d) water-soluble sweeteners derived from naturally occurring water-soluble sweeteners, such as steviosides, chlorinated derivatives of ordinary sugar (sucrose), e.g., chlorodeoxysugar derivatives such as derivatives of chlorodeoxysucrose or chlorodeoxygalactosucrose, known, for example, under the product designation of Sucralose or Splenda; examples of chlorodeoxysucrose and chlorodeoxygalactosucrose derivatives include but are not limited to: 1-chloro-1′-deoxysucrose; 4-chloro-4-deoxy-alpha-D-galactopyranosyl-alpha-D-fructofuranoside, or 4-chloro-4-deoxygalactosucrose; 4-chloro-4-deoxy-alpha-D-galactopyranosyl-1-chloro-1-deoxy-beta-D-fructo-furanoside, or 4,1′-dichloro-4,1′-dideoxygalactosucrose; 1′,6′-dichloro-1′,6′-dideoxysucrose; 4-chloro-4-deoxy-alpha-D-galactopyranosyl-1,6-dichloro-1,6-dideoxy-beta-D-fructofuranoside, or 4,1′,6′-trichloro-4,1′,6′-trideoxygalactosucrose; 4,6-dichloro-4,6-dideoxy-alpha-D-galactopyranosyl-6-chloro-6-deoxy-beta-D-fructofuranoside, or 4,6,6′-trichloro-4,6,6′-trideoxygalactosucrose; 6,1′,6′-trichloro-6,1′,6′-trideoxysucrose; 4,6-dichloro-4,6-dideoxy-alpha-D-galacto-pyranosyl-1,6-dichloro-1,6-dideoxy-beta-D-fructofuranoside, or 4,6,1′,6′-tetrachloro4,6,1′,6′-tetradeoxygalacto-sucrose; 4,6,1′,6′-tetradeoxy-sucrose, or a combination of at least two of the foregoing;

(e) protein based sweeteners such as thaumaoccous danielli, talin, or a combination of at least two of the foregoing;

(f) amino acid based sweeteners; and

(g) the sweetener monatin (2-hydroxy-2-(indol-3-ylmethyl)-4-aminoglutaric acid) and its derivatives.

The high intensity sweetener can be used in a variety of distinct physical forms, for example those known in the art to provide an initial burst of sweetness and/or a prolonged sensation of sweetness. Without being limited thereto, such physical forms include free forms (e.g., spray dried or powdered), beaded forms, encapsulated forms, or a combination of at least two of the foregoing forms.

In one embodiment, the high intensity sweetener composition includes neotame (N—[N-(3,3-dimethylbutyl)-L-α-aspartyl]-L-phenylalanine-1-methyl ester). Neotame is about 8,000 to about 10,000 times sweeter then sucrose on a per weight basis. In another embodiment, the high intensity sweetener composition includes sucralose (1,6-dichloro-1,6-dideoxy-β-D-fructo-furanosyl 4-chloro-4-deoxy-α-D-galactopyranoside). Sucralose is about 600 times sweeter than sucrose on a per weight basis. The high intensity sweetener can also be a combination comprising neotame and sucralose. In a specific embodiment, the flavor-enhancing composition for a comestible product comprises dextromethorphan, menthol, and neotame or sucralose or a combination of neotame and sucralose.

A wide variety of one or more conventional additives can be used with the flavor-enhancing compositions, including flavor modulators or potentiators, flavorants, additional sweeteners, coloring agents, additional medicaments, breath fresheners, mineral adjuvants, bulking agents, acidulants, buffering agents, thickeners, additional coolants, mouth moisteners, antioxidants (e.g., butylated hydroxytoluene (BHT), butylated hydroxyanisole (BHA), or propyl gallate), preservatives, and the like. Some of these additives may serve more than one purpose. For example, an additional sweetener, e.g., sucrose, sorbitol or other sugar alcohol, or combinations of the foregoing additional sweeteners, may also function as a bulking agent. A combination of at least two of the foregoing additives can be used.

In a comestible, a sweet taste can come from flavor modulators or potentiators and/or from flavorants as well as from sweeteners. Flavor potentiators can consist of materials that intensify, supplement, modify or enhance the taste or aroma perception of an original material without introducing a characteristic taste or aroma perception of their own. Flavor modulators may impart a characteristic of their own that complements or negates a characteristic of another component. In some embodiments, flavor modulators or potentiators designed to intensify, supplement, modify, or enhance the perception of flavor, sweetness, tartness, umami, kokumi, saltiness, and combinations of at least two of the foregoing can be included. Thus, the addition of flavor modulators or potentiators can impact the overall taste of the comestible. For example, flavors can be compounded to have additional sweet notes by the inclusion of flavor modulators or potentiators, such as vanilla, vanillin, ethyl maltol, furfual, ethyl propionate, lactones, or a combination of at least two of the foregoing flavor agents.

Exemplary flavor modulators or potentiators include monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, neotame, thaumatin, neohesperidin dihydrochalcone, chlorogenic acid, tagatose, trehalose, maltol, ethyl maltol, quercetin, vanilla extract (e.g., in ethyl alcohol), vanilla oleoresin, vanillin, sugar beet extract (alcoholic extract), sugarcane leaf essence (alcoholic extract), compounds that respond to G-protein coupled receptors (T2Rs and T1Rs), or a combination of at least two of the foregoing. In some embodiments, sugar acids, quercetin, sodium chloride, potassium chloride, sodium acid sulfate, or a combination of at least two of the foregoing are used. In other embodiments, glutamates such as monosodium glutamate, monopotassium glutamate, hydrolyzed vegetable protein, hydrolyzed animal protein, yeast extract, or a combination of at least two of the foregoing are included. Further examples include adenosine monophosphate (AMP), glutathione, and nucleotides such as inosine monophosphate, disodium inosinate, xanthosine monophosphate, guanylate monophosphate, compositions comprising 5′-nucleotides such as those disclosed in U.S. 2006/0078972 to Noordam et al, or a combination of at least two of the foregoing. Further examples of flavor potentiator compositions that impart kokumi are also included in U.S. Pat. No. 5,679,397 to Kuroda et al. “Kokumi” refers to materials that impart “mouthfulness” and “good body”. Combinations comprising one or more of the above flavor modulators and potentiators may be used.

In one embodiment, a composition comprises menthol and a bitterness-reducing amount of one or more of the flavor modulators and potentiators described in the preceding paragraph. The composition need not comprise a medicament, a non-menthol physiological cooling agent, or a high intensity sweetener, but such components are optionally included. In some embodiments, the flavor modulator or potentiator is sodium chloride, monosodium glutamate, quercetin, adenosine monophosphate, inosine monophosphate, guanylate monophosphate, an edible salt of one of the foregoing, or a combination thereof. Surprisingly, it has been found that such compounds are capable of use at levels that reduce the unwanted bitterness of menthol without introducing an unwanted salty flavor. This is particularly surprising in the case of quercetin, which itself has an unwanted bitterness.

Further examples of flavor potentiators include sweetness potentiators. Sweetness potentiators include monoammonium glycyrrhizinate, licorice glycyrrhizinates, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, pyridinium-betain compounds, sugar beet extract, neotame, thaumatin, neohesperidin dihydrochalcone, hydroxybenzoic acids, 2-hydroxybenzoic acid (2-HB), 3-hydroxybenzoic acid (3-HB), 4-hydroxybenzoic acid (4-HB), 2,3-dihydroxybenzoic acid (2,3-DHB), 2,4-dihydroxybenzoic acid (2,4-DHB), 2,5-dihydroxybenzoic acid (2,5-DHB), 2,6-dihydroxybenzoic acid (2,6-DHB), 3,4-dihydroxybenzoic acid (3,4-DHB), 3,5-dihydroxybenzoic acid (3,5-DHB), 2,3,4-trihydroxybenzoic acid (2,3,4-THB), 2,4,6-trihydroxybenzoic acid (2,4,6-THB), 3,4,5-trihydroxybenzoic acid (3,4,5-THB), 4-hydroxyphenylacetic acid, 2-hydroxyisocaproic acid, 3-hydroxycinnamic acid, 3-aminobenzoic acid, 4-aminobenzoic acid, 4-methoxysalicylic acid, 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl)ethanone, 2,4-dihydroxy-N-[(4-hydroxy-3-methoxyphenyl)methyl]benzamide, 2,4,6-trihydroxy-N-[(4-hydroxy-3-methoxyphenyl)methyl]benzamide, 2-hydroxy-N-[(4-hydroxy-3-methoxyphenyl)methyl]benzamide, 4-hydroxy-N-[(4-hydroxy-3-methoxyphenyl)methyl]benzamide, 2,4-dihydroxy-N-[(4-hydroxy-3-methoxyphenyl)methyl]benzamide, 2,4-dihydroxy-N-[2-(4-hydroxy-3-methoxyphenyl)ethyl]benzamide, N-[(3-ethoxy-4-hydroxyphenyl)methyl]-2,4-dihydroxy-benzamide, N-[(3,4-dihydroxyphenyl)methyl]-2,4-dihydroxy-benzamide, tagatose, trehalose, maltol, ethyl maltol, vanilla extract, vanilla oleoresin, vanillin, sugar beet extract (alcoholic extract), sugarcane leaf essence (alcoholic extract), compounds that respond to G-protein coupled receptors (T2Rs and T1Rs), edible salts of the foregoing, and combinations of at least two of the foregoing. These and other sweetness potentiators are described in, for example, International Patent Application Nos. WO 2006/024587 A1 and WO 2006/106023 A1 of Ley et al.

Flavorants that can be used include those artificial and natural flavors known in the art, for example synthetic flavor oils, natural flavoring aromatics and/or oils, oleoresins, extracts derived from plants, leaves, flowers, fruits, and the like, and combinations comprising at least one of the foregoing flavorants. Nonlimiting representative flavors include oils such as spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, cassia oil, and citrus oils including lemon, orange, lime, grapefruit, vanilla, fruit essences, including apple, pear, peach, grape, strawberry, raspberry, blackberry, cherry, plum, pineapple, apricot, banana, melon, tropical fruit, mango, mangosteen, pomegranate, papaya, honey lemon, and the like, or a combination of at least two of the foregoing flavorants. Specific flavorants are mints such as peppermint, spearmint, artificial vanilla, cinnamon derivatives, and various fruit flavors.

Other types of flavorants include various aldehydes and esters such as cinnamyl acetate, cinnamaldehyde, citral diethylacetal, dihydrocarvyl acetate, eugenyl formate, p-methylamisol, acetaldehyde (apple), benzaldehyde (cherry, almond), anisic aldehyde (licorice, anise), cinnamic aldehyde (cinnamon), citral, i.e., alpha-citral (lemon, lime), neral, i.e., beta-citral (lemon, lime), decanal (orange, lemon), ethyl vanillin (vanilla, cream), heliotrope, i.e., piperonal (vanilla, cream), vanillin (vanilla, cream), alpha-amyl cinnamaldehyde (spicy fruity flavors), butyraldehyde (butter, cheese), valeraldehyde (butter, cheese), citronellal (modifies, many types), decanal (citrus fruits), aldehyde C-8 (citrus fruits), aldehyde C-9 (citrus fruits), aldehyde C-12 (citrus fruits), 2-ethyl butyraldehyde (berry fruits), hexenal, i.e., trans-2 (berry fruits), tolyl aldehyde (cherry, almond), veratraldehyde (vanilla), 2,6-dimethyl-5-heptenal, i.e., melonal (melon), 2,6-dimethyloctanal (green fruit), and 2-dodecenal (citrus, mandarin).

The flavoring agent can be used in liquid or solid form. When used in solid (dry) form, suitable drying means such as spray drying the oil may be used. Alternatively, the secondary flavoring agent can be encapsulated, absorbed onto water soluble materials by means known in the art, for example cellulose, starch, sugar, maltodextrin, gum arabic, and the like. In some embodiments, the secondary flavoring agents can be used in physical forms effective to provide an initial burst of flavor or a prolonged sensation of flavor.

In addition to the high intensity sweetener, further sweeteners that can be used include natural and artificial water-soluble sweeteners, including water-soluble sweeteners derived from naturally occurring water-soluble sweeteners, dipeptide based sweeteners, protein based sweeteners, sugar alcohols such as sorbitol, mannitol, maltitol, isomalt, lactitol, hydrogenated starch hydrolysates, maltitol syrups, xylitol, erythritol, and combinations of at least two of the foregoing additional sweeteners. Representative categories and examples of suitable additional sweeteners include mogroside, monosaccharides, disaccharides and polysaccharides such as xylose, ribulose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, and polyols (e.g., glycerol, sorbitol, maltitol, maltitol syrup, mannitol, isomalt, erythritol, xylitol, hydrogenated starch hydrolysates, polyglycitol syrups, polyglycitol powders, lactitol), or a combination of at least two of the foregoing.

Coloring can be used in amounts effective to produce a desired color for the comestible. Suitable coloring agents include pigments, which may be incorporated in amounts up to about 6 wt % (weight %) by weight of the comestible. For example, titanium dioxide may be incorporated in amounts up to about 2 wt %, and specifically less than about 1 wt % by weight of the comestible. Suitable coloring agents also include natural food colors and dyes suitable for food, drug, and cosmetic applications. Suitable colors include annatto extract (E160b), bixin, norbixin, astaxanthin, dehydrated beets (beet powder), beetroot red/betanin (E162), ultramarine blue, canthaxanthin (E161g), cryptoxanthin (E161c), rubixanthin (E161d), violanxanthin (E161e), rhodoxanthin (E161f), caramel (E150(a-d)), β-apo-8′-carotenal (E160e), β-carotene (E160a), alpha carotene, gamma carotene, ethyl ester of beta-apo-8 carotenal (E160f), flavoxanthin (E161a), lutein (E161b), cochineal extract (E120), carmine (E132), carmoisine/azorubine (E122), sodium copper chlorophyllin (E141), chlorophyll (E140), toasted partially defatted cooked cottonseed flour, ferrous gluconate, ferrous lactate, grape color extract, grape skin extract (enocianina), anthocyanins (E163), haematococcus algae meal, synthetic iron oxide, iron oxides and hydroxides (E172), fruit juice, vegetable juice, dried algae meal, tagetes (Aztec marigold) meal and extract, carrot oil, corn endosperm oil, paprika, paprika oleoresin, phaffia yeast, riboflavin (E101), saffron, titanium dioxide, turmeric (E100), turmeric oleoresin, amaranth (E123), capsanthin/capsorbin (E160c), lycopene (E160d), FD&C blue #1, FD&C blue #2, FD&C green #3, FD&C red #3, FD&C red #40, FD&C yellow #5 and FD&C yellow #6, tartrazine (E102), quinoline yellow (E104), sunset yellow (E110), ponceau (E124), erythrosine (E127), patent blue V (E131), titanium dioxide (E171), aluminium (E173), silver (E174), gold (E175), pigment rubine/lithol rubine BK (E180), calcium carbonate (E170), carbon black (E153), black PN/brilliant black BN (E151), green S/acid brilliant green BS (E142), or a combination of at least two of the foregoing. In some embodiments, certified colors can include FD&C aluminum lakes, or a combination of at least two of the foregoing colors.

Additional optional medicaments can be included in the comestible product. Nonlimiting illustrative categories and specific examples include antacids, antinauseants, antifungal agents, chemotherapeutics, diuretics, psychotherapeutic agents, cardiovascular agents, various alkaloids, laxatives, appetite suppressants, ACE-inhibitors, anti-asthmatics, anti-cholesterolemics, anti-depressants, anti-diarrhea preparations, anti-hypertensives, anti-lipid agents, acne drugs, amino acid preparations, anti-uricemic drugs, anabolic preparations, appetite stimulants, bone metabolism regulators, contraceptives, endometriosis management agents, enzymes, erectile dysfunction therapies such as sildenafil citrate, fertility agents, gastrointestinal agents, homeopathic remedies, hormones, motion siclness treatments, muscle relaxants, osteoporosis preparations, oxytocics, parasympatholytics, parasympathomimetics, prostaglandins, respiratory agents, sedatives, smoking cessation aids such as bromocryptine or nicotine, tremor preparations, urinary tract agents, anti-ulcer agents, anti-emetics, hyper- and hypo-glycemic agents, thyroid and anti-thyroid preparations, terine relaxants, erythropoietic drugs, mucolytics, DNA and genetic modifying drugs, and nutritional supplements, including nutraceuticals, micronutrients, vitamins and co-enzymes. Combinations of the foregoing types of optional medicaments can be used.

Exemplary antacids include cimetidine, ranitidine, nizatidine, famotidine, omeprazole, bismuth antacids, metronidazole antacids, tetracycline antacids, clartlromycin antacids, hydroxides of aluminum, magnesium, sodium bicarbonates, calcium bicarbonate and other carbonates, silicates, phosphates, or a combination of at least two of the foregoing.

Antifungal agents include, for example, ketoconazole, fluconazole, nystatin, itraconazole, clomitrazole, natamycin, econazole, isoconazole, oxiconazole, thiabendazole, tiaconazole, voriconazole, terbinafine, amorolfine, micfungin, amphotericin B, or a combination of at least two of the foregoing.

Exemplary chemotherapeutics agents include cisplatin (CDDP), procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxol, transplatinum, 5-fluorouracil, vincristin, vinblastin and methotrexate or any analog or derivative variant thereof, or a combination of at least two of the foregoing.

Exemplary diuretics include but are not limited to acetazolamide, dichlorphenamide, methazolamide, furosemide, bumetanide, ethacrynic acid torsemide, azosemide, muzolimine, piretanide, tripamide, bendroflumethiazide, benzthiazide, chlorothiazide, hydrochlorothiazide, hydroflumethiazide, methyclothiazide, polythiazide, trichlormethiazide, indapamide, metolazone, quinethazone, amiloride, triamterene, sprionolactone, canrenone, potassium canrenoate, or a combination of at least two of the foregoing.

Exemplary psychotherapeutic agents include thorazine, serentil, mellaril, millazine, tindal, permitil, prolixin, trilafon, stelazine, suprazine, taractan, navan, clozaril, haldol, halperon, loxitane, moban, orap, risperdal, alprazolam, chlordiaepoxide, clonezepam, clorezepate, diazepam, halazepam, lorazepam, oxazepam, prazepam, buspirone, elvavil, anafranil, adapin, sinequan, tofranil, surmontil, asendin, norpramin, pertofrane, ludiomil, pamelor, vivactil, prozac, luvox, paxil, zoloft, effexor, welibutrin, serzone, desyrel, nardil, parnate, eldepryl, or a combination of at least two of the foregoing.

Exemplary cardiovascular agents include nitroglycerin, isosorbide dinitrate, sodium nitroprisside, captopril, enalapril, enalaprilat, quinapril, lisinopril, ramipril, losartan, amrinone, lirinone, vesnerinone, hydralazine, nicorandil, prozasin, doxazosin, bunazosin, tamulosin, yohimbine, propanolol, metoprolol, nadolol, atenolol, timolol, esmolol, pindolol, acebutolol, labetalol, phentolamine, carvedilol, bucindolol, verapamil, nifedipine, amlodipine dobutamine, or a combination of at least two of the foregoing.

Exemplary appetite suppressants include benzphetamine, diethylpropion, mazindol, phendimetrazine, phentermine, hoodia, ephedra, and caffeine. Additional appetite suppressant are commercially under the following trade names: Adipex, Adipost, Bontril PDM, Bontril Slow Release, Didrex, Fastin, lonamin, Mazanor, Melfiat, Obenix, Phendiet, Phendiet-105, Phentercot, Phentride, Plegine, Prelu-2, Pro-Fast, PT 105, Sanorex, Tenuate, Sanorex, Tenuate, Tenuate Dospan, Tepanil Ten-Tab, Teramine, Zantryl or a combination of at least two of the foregoing.

Nutraceuticals and micronutrients can include herbs and botanicals such as aloe, bilberry, bloodroot, calendula, capsicum, chamomile, cat's claw, echinacea, garlic, ginger, ginko, goldenseal, various ginseng, green tea, golden seal, guarana, kava kava, lutein, nettle, passionflower, rosemary, saw palmetto, St. John's wort, thyme, and valerian. Also included are mineral supplements such as calcium, copper, iodine, iron, magnesium, manganese, molybdenum, phosphorous, zinc, and selenium. Other nutraceuticals that also can be added include fructooligosaccharides, glucosamine, grapeseed extract, cola extract, guarana, ephedra, inulin, phytosterols, phytochemicals, catechins, epicatechin, epicatechin gallate, epigallocatechin, epigallocatechin gallate, isoflavones, lecithin, lycopene, oligofructose, polyphenols, flavanoids, flavanols, flavonols, and psyllium as well as weight loss agents such as chromium picolinate and phenylpropanolamine. Exemplary vitamins and co-enzymes include water or fat soluble vitamins such as thiamin, riboflavin, nicotinic acid, pyridoxine, pantothenic acid, biotin, folic acid, flavin, choline, inositol and paraminobenzoic acid, carnitine, vitamin C, vitamin D and its analogs, vitamin A and the carotenoids, retinoic acid, vitamin E, vitamin K, vitamin B₆, and vitamin B₁₂. Combinations comprising at least one of the foregoing nutraceuticals can be used.

Specific optional, additional medicaments that can be used include caffeine, cimetidine, ranitidine, famotidine, omeprazole, dyclonine, nicotine, or a combination of at least two of the foregoing.

Exemplary breath fresheners include to zinc citrate, zinc acetate, zinc fluoride, zinc ammonium sulfate, zinc bromide, zinc iodide, zinc chloride, zinc nitrate, zinc fluorosilicate, zinc gluconate, zinc tartarate, zinc succinate, zinc formate, zinc chromate, zinc phenol sulfonate, zinc dithionate, zinc sulfate, silver nitrate, zinc salicylate, zinc glycerophosphate, copper nitrate, chlorophyll, copper chlorophyll, chlorophyllin, hydrogenated cottonseed oil, chlorine dioxide, beta cyclodextrin, zeolite, silica-based material, carbon-based material, enzymes such as laccase, or a combination of at least two of the foregoing. Breath fresheners can include essential oils as well as various aldehydes and alcohols. Essential oils used as breath fresheners can include oils of spearmint, peppermint, wintergreen, sassafras, chlorophyll, citral, geraniol, cardamom, clove, sage, carvacrol, eucalyptus, cardamom, magnolia bark extract, marjoram, cinnamon, lemon, lime, grapefruit, orange, or a combination of at least two of the foregoing. Aldehydes such as cinnamic aldehyde and salicylaldehyde can be used. Additionally, chemicals such as menthol, carvone, iso-garrigol, and anethole can function as breath fresheners.

Exemplary mouth moisteners include saliva stimulators such as acids and salts including acetic acid, adipic acid, ascorbic acid, butyric acid, citric acid, formic acid, fumaric acid, glyconic acid, lactic acid, phosphoric acid, malic acid, oxalic acid, succinic acid, and tartaric acid. Mouth moisteners can include hydrocolloid materials that hydrate and may adhere to oral surface to provide a sensation of mouth moistening. Hydrocolloid materials can include naturally occurring materials such as plant exudates, seed gums, and seaweed extracts or they can be chemically modified materials such as cellulose, starch, or natural gum derivatives. Furthermore, hydrocolloid materials can include pectin, gum arabic, acacia gum, alginates, agar, carageenans, guar gum, xanthan gum, locust bean gum, gelatin, gellan gum, galactomannans, tragacanth gum, karaya gum, curdlan, konjac, chitosan, xyloglucan, beta glucan, furcellaran, gum ghatti, tamarin, and bacterial gums. Mouth moisteners can include modified natural gums such as propylene glycol alginate, carboxymethyl locust bean gum, low methoxyl pectin, or a combination of at least two of the foregoing. Modified celluloses can be included such as microcrystalline cellulose, carboxymethylcellulose (CMC), methylcellulose (MC), hydroxypropylmethylcellulose (HPCM), hydroxypropylcellulose (MPC), or a combination of at least two of the foregoing mouth moisteners.

Similarly, humectants, which can provide a perception of mouth hydration, can be included. Such humectants can include glycerol, sorbitol, polyethylene glycol, erythritol, xylitol, or a combination of at least two of the foregoing. Additionally, in some embodiments, fats can provide a perception of mouth moistening. Such fats can include medium chain triglycerides, vegetable oils, fish oils, mineral oils, or a combination of at least two of the foregoing.

Suitable acidulants illustratively include acetic, citric, fumaric, hydrochloric, lactic and nitric acids as well as sodium citrate, sodium bicarbonate and carbonate, sodium or potassium phosphate and magnesium oxide, potassium metaphosphate, sodium acetate, or a combination of at least two of the foregoing acidulants.

Exemplary buffering agents include sodium bicarbonate, sodium phosphate, sodium hydroxide, ammonium hydroxide, potassium hydroxide, sodium stannate, triethanolamine, citric acid, hydrochloric acid, sodium citrate, or a combination of at least two of the foregoing buffering agents.

The relative amounts of each of the components of the flavor-enhancing concentration will depend on the particular comestible, medicament, coolant, high intensity sweetener, and optional additives, as well as the desired flavor, and are readily determined by one of ordinary skill in the art without undue experimentation, using the guidelines provided below.

As mentioned above, each medicament is present in the comestible in an amount that will provide the desired dose per unit of the comestible. Depending upon the therapeutic dosage recommended or permitted, the medicament can be present in an amount of about 0.00001 wt % (weight %) to about 2 wt % of the comestible product. In another embodiment, the medicament is present in an amount of about 0.00025 wt % to about 1 wt %, more specifically about 0.01 wt % to about 1 wt %, each based on the total weight of the comestible product.

A menthyl-containing coolant (or combination thereof) is present in the comestible in an amount effective to provide flavor enhancement, for example an amount of about 0.00001 wt % to about 5 wt % of the total weight of the comestible product. In another embodiment, a menthyl-containing coolant is present in an amount of about 0.00025 wt % to about 3 wt %, specifically about 0.001 wt % to about 1 wt %, each based on the total weight of the comestible product.

A high intensity sweetener (or combination thereof) is present in the comestible in an amount effective to provide flavor enhancement, for example about 0.0001 wt % to about 2 wt %, specifically about 0.005 wt % to about 1 wt %, more specifically about 0.025 wt % to about 0.5 wt %, each based on the total weight of the comestible product.

The constituent components of the flavor-enhancing composition (i.e., the medicament, coolant, high intensity sweetener, and other optional additive(s)) can be added together, separately, or at different stages during manufacture of the comestible product. Alternatively, the flavor-enhancing compositions can be prepared in the form of a concentrate. Methods for the manufacture of concentrates are known in the art, and generally comprise admixture of the desired ingredients with or without a diluent or carrier, such as water. Once prepared, the concentrate can be stored for future use. The concentrate can also be formulated with conventional additives as described above.

The relative amounts of each component of the concentrate will depend on the desired final amounts in the comestible product, the presence of any optional additives, or the use of a diluent. The relative amounts can be readily determined by one of ordinary skill in the art without undue experimentation, using the below guidelines.

For example, in one embodiment, the flavor-enhancing composition comprises about 1 wt % to about 60 wt % of a medicament, about 5 wt % to about 95 wt % of a menthyl-containing coolant, and about 10 wt % to about 80 wt. % of a high intensity sweetener, each based on the total weight of the composition.

In a specific embodiment, the concentrate comprises about 1 wt % to about 60 wt % of dextromethorphan; about 1 wt % to about 95 wt %, specifically about 5 wt % to about 90 wt %, more specifically about 10 to about 90 wt % of menthol; about 0.01 wt % to about 15 wt %, specifically about 0.05 wt % to about 10 wt % of WS-3; about 0.01 wt % to about 15 wt %, specifically about 0.1 wt % to about 5 wt %, of menthyl glutarate esters; and about 0.01 wt % to about 20 wt %, specifically about 0.05 wt % to about 15 wt %, more specifically about 0.1 wt % to about 10 wt % of neotame, or about 1 to about 80 wt %, specifically about 1 wt % to about 50 wt %, more specifically about 5 to about 40 wt % sucralose.

In some embodiments, the concentrate includes an acidulant, a buffering agent, or a combination of an acidulant and a buffering agent. The acidulant can be used in amounts of about zero wt % to about 60.0 wt %, specifically about 10 wt % to about 50 wt % of the total weight of the concentrate. Similarly, the buffering agent can be used in amounts of about zero wt % to about 60 wt %, specifically about 10 wt % to about 50 wt % of the total weight of the concentrate.

One embodiment is a flavor enhancing composition comprising a dextromethorphan, menthol, and neotame or sucralose or both.

One embodiment is a flavor-enhancing composition for a comestible product, comprising: about 1 to about 50 weight percent sucralose; about 0.01 to about 15 weight percent neotame; about 0.1 to about 50 weight percent sodium citrate; about 1 to about 50 weight percent acidulant; about 1 to about 90 weight percent menthol; about 0.01 to about 20 weight percent N-ethyl-p-menthane-3-carboxamide, menthyl glutarate, or a combination thereof; and about 0.01 to about 10 weight percent dextromethorphan; wherein all weight percents are based on the total weight of the flavor-enhancing composition.

The flavor-enhancing compositions can be used to prepare a wide variety of comestible products, and the present invention extends to methods of making the comestible product. As used herein, a “comestible product” broadly includes all products that are ingestible, whether or not they provide nutritive value, and includes, for example, beverages, foods in all forms (including forms requiring reconstitution), jellies, condiments, confectioneries, extracts, nutraceuticals, gelatins, gums, tablets, lozenges, drops, emulsions, elixirs, sprays, gels, and syrups, pharmaceutical compositions administered orally, nasally, and the like, as well as hygienic products such as toothpastes, dental lotions, or mouth washes.

A comestible product is made by admixing the concentrate or the individual ingredients of the flavor-enhancing composition with the other ingredients of the final desired composition. Other ingredients will usually be incorporated into the composition as dictated by the nature of the desired composition as well known to those of ordinary skill in the art. The ultimate consumable product or confectionery composition compositions are readily prepared using methods generally known in the food technology and pharmaceutical arts. While it is often convenient to manufacture such products using a concentrate, is it also within the scope of the present invention to add the constituent elements of the concentrate (i.e., the dextromethorphan, menthol, sweetener, and other optional additive(s)) separately or at different stages during manufacture of the product.

In one embodiment, the concentrate is present in amounts of about 0.001 wt % to about 40.0 wt % of the total weight of the comestible product. In another embodiment, the concentrate is used in amounts of about 0.01 wt % to about 20 wt % of the total weight of the comestible product. In another embodiment, the concentrate is used in amounts of about 0.05 wt % to about 10 wt % of the total weight of the comestible product.

The flavor-enhancing composition can be of particular utility in the preparation of dosage delivery systems with confectionery components, including, for example, compressed tablets such as mints, hard boiled candies, chocolates, chocolate-containing products, nutrient bars, nougats, gels, centerfill confections, fondants, panning goods, consumable thin films, and other confectionery formats. Confectioneries have been classified as either “hard” or “soft” confectionery items. In one embodiment the flavor-enhancing composition is used in a confectionery format, in particular a hard confectionery such as a lozenge. In another embodiment, the flavor-enhancing composition is used in a chewing gum. The flavor-enhancing compositions can be incorporated into an otherwise conventional hard or soft confectionery format using standard techniques and equipment known to those of ordinary skill in the art.

In general, a hard confectionery has a base composed of a mixture of sugar or sugar alcohols and other carbohydrate bulking agents, kept in an amorphous or glassy condition. This form is considered a solid syrup of sugars or sugar alcohols generally having from about 0.5 wt % to about 1.5 wt % moisture. Such materials normally contain up to about 92 wt % corn syrup, up to about 55 wt % sugar and from about 0.1 wt % to about 5 wt % water, all based on the weight of the base. The syrup component can be prepared from corn syrups high in fructose, but may include other materials.

In some embodiments, the hard confectioneries are prepared using conventional methods and equipment, such as fire cookers, vacuum cookers, or scraped-surface cookers (also referred to as high speed atmospheric cookers). When using a fire cooker, the desired quantity of carbohydrate bulking agent is dissolved in water by heating the agent in a kettle until the bulking agent dissolves. Additional bulking agent may then be added and cooking continued until a final temperature of, for example, 145° C. to 156° C. is achieved. The batch is then cooled and worked as a plastic-like mass to incorporate additives separately or in the form of one or more concentrates.

In vacuum cookers, a carbohydrate-bulking agent is boiled to about 125° to about 132° C., vacuum is applied, and additional water is boiled off without extra heating. When cooking is complete, the mass is a semi-solid and has a plastic-like consistency. At this point, additives, separately or in the form of one or more concentrates are admixed in the mass by routine mechanical mixing operations.

A high-speed atmospheric cooker uses a heat exchanger surface. A film of a hard confectionery composition is spread on a heat exchange surface, rapidly heated to a suitable temperature, for example 165° to 170° C., and then rapidly cooled, for example to 100° to 120° C. Additives, separately or in the form of one or more concentrates can then be worked into the plastic mass.

In the foregoing methods, the additive(s) are specifically mixed for a time effective to provide a uniform distribution of the materials, for example about 4 to about 10 minutes. Once the hard confectionery mass has been properly tempered, it can be cut into workable portions or formed into desired shapes as is known in the art.

Compressed tablet confectionery formats, in contrast, are formed into structures under pressure. These confections generally contain sugars or sugar alcohols in amounts up to about 95% by weight of the composition, tablet excipients such as binders and lubricants, as well as additives.

The preparation of soft confectionery such as nougat, involves conventional methods, such as the combination of two primary components, namely (1) a high boiling syrup such as a corn syrup, hydrogenated starch hydrolysate or the like, and (2) a relatively light textured frappe. The high boiling syrup, or “bob syrup” of the soft confectionery is relatively viscous and has a higher density than the frappe component, and frequently contains a substantial amount of carbohydrate bulking agent such as a hydrogenated starch hydrolysate. The frappe is generally prepared from egg albumin, gelatin, vegetable proteins, such as soy-derived compounds, sugarless milk derived compounds, such as milk proteins, and mixtures thereof. The frappe is generally relatively light, and may, for example, range in density from about 0.5 to about 0.7 grams/cc. Conventionally, the final nougat composition is prepared by the addition of the bob syrup to the frappe under agitation, to form the basic nougat mixture. For example, the frappe component is prepared first and thereafter the syrup component is slowly added under agitation at a suitable temperature, for example at least about 65° C., and specifically at least about 100° C. After formation of a uniform mixture, the mixture is cooled, for example to below about 80° C., at which point additional ingredients such as flavoring, additional carbohydrate bulking agent, coloring agents, preservatives, medicaments, and the like may be added with further mixing. The mixture is then formed into suitable confectionery shapes.

The flavor-enhancing composition is also useful in the manufacture of chewing gums, including both chewing gum and bubble gum formulations. With regard to chewing gum compositions, such compositions contain a gum base, the flavor-enhancing composition, and various additives.

The gum base can vary greatly depending upon various factors such as the type of base desired, the consistency of gum desired, and the other components used in the composition to make the final chewing gum product. The gum base may be any water-insoluble gum base known in the art, and includes those gum bases utilized for chewing gums and bubble gums. Illustrative examples of suitable polymers in gum bases include both natural and synthetic elastomers and rubbers, for example, substances of vegetable origin such as chicle, crown gum, nispero, rosadinha, jelutong, perillo, niger gutta, tunu, balata, gutta-percha, lechi-capsi, sorva, gutta kay, and the like. Synthetic elastomers such as butadiene-styrene copolymers, polyisobutylene, isobutylene-isoprene copolymers, polyethylene, a combination thereof, and the like are also useful. The gum base may include a non-toxic vinyl polymer, such as polyvinyl acetate and its partial hydrolysate, polyvinyl alcohol, or a combination of at least two of the foregoing. When utilized, the molecular weight of the vinyl polymer may range from about 3,000 up to and including about 94,000.

The amount of gum base employed will vary greatly depending upon various factors such as the type of base used, the consistency of the gum desired, and the other components used in the composition to make the final chewing gum product. In general, the gum base will be present in amounts of about 5 wt % to about 94 wt % of the final chewing gum composition, or in amounts of about 15 wt % to about 45 wt %, and more specifically in amounts of about 15 wt % to about 35 wt %, and most specifically about 20 wt % to about 30 wt % of the chewing gum product.

The gum base composition may contain conventional elastomer solvents to aid in softening the elastomer base component, for example trepanned resins such as polymers of alpha-pinene or beta-pinene, methyl, glycerol or pentaerythritol esters of rosins or modified rosins and gums, such as hydrogenated, dimerized or polymerized rosins, or combinations comprising at least one of the foregoing resins, the pentaerythritol ester of partially hydrogenated wood or gum rosin, the pentaerythritol ester of wood or gum rosin, the glycerol ester of wood rosin, the glycerol ester of partially dimerized wood or gum rosin, the glycerol ester of polymerized wood or gum rosin, the glycerol ester of tall oil rosin, the glycerol ester of wood or gum rosin, the partially hydrogenated wood or gum rosin, the partially hydrogenated methyl ester of wood or rosin, and the like. The elastomer solvent can be used in amounts of about 5 wt % to about 75 wt %, of the gum base, and specifically about 45 wt % to about 70 wt % of the gum base.

Conventional additives can be included in the gum base in effective amounts such as plasticizers or softeners such as lanolin, palmitic acid, oleic acid, stearic acid, sodium stearate, potassium stearate, glyceryl triacetate, glyceryl lecithin, glyceryl monostearate, propylene glycol monostearate, acetylated monoglyceride, glycerine, and the like, to obtain a variety of desirable textures and consistency properties. Waxes, for example, natural and synthetic waxes, hydrogenated vegetable oils, petroleum waxes such as polyurethane waxes, polyethylene waxes, paraffin waxes, microcrystalline waxes, fatty waxes, sorbitan monostearate, tallow, propylene glycol, and the like can also be incorporated into the gum base to obtain a variety of desirable textures and consistency properties. These additives are generally used in amounts of up to about 30 wt % of the gum base, specifically about 3 wt % to about 20 wt % of the gum base.

The gum base can include effective amounts of mineral adjuvants such as calcium carbonate, magnesium carbonate, alumina, aluminum hydroxide, aluminum silicate, talc, tricalcium phosphate, tricalcium phosphate and the like, which can serve as fillers and textural agents. These fillers or adjuvants can be used in the gum base in various amounts. Specifically the amount of filler, when used, will be present in an amount of greater than about 0 wt % to about 60 wt % of the chewing gum base.

Examples of other useful additives include emulsifiers, such as lecithin and glyceryl monostearate, thickeners, used alone or in combination with other softeners, such as methyl cellulose, alginates, carrageenan, xanthan gum, gelatin, carob, tragacanth, locust bean, and carboxymethylcellulose, acidulants such as malic acid, adipic acid, citric acid, tartaric acid, fumaric acid, and mixtures thereof, and fillers, such as those discussed above under the category of mineral adjuvants. Bulking agents (carriers, extenders) suitable for use include sweetening agents selected from the group consisting of monosaccharides, disaccharides, polysaccharides, sugar alcohols, and mixtures thereof, polydextrose; maltodextrins; minerals, such as calcium carbonate, talc, titanium dioxide, dicalcium phosphate, and the like. Bulking agents may be used in amounts up to about 90 wt % of the final gum composition, specifically about 40 wt % to about 70 wt %, and about 50 wt % to about 65 wt % of the gum composition being most preferred.

The flavor-enhancing composition can be incorporated into an otherwise conventional chewing gum composition using standard techniques and equipment. In one exemplary process, a gum base is heated to a temperature sufficiently high to soften the base without adversely effecting the physical and chemical make up of the base, which will vary depending upon the composition of the gum base used, and is readily determined by those skilled in the art without undue experimentation. For example, the gum base can be conventionally melted to about 60° C. to about 120° C. for a period of time sufficient to render the base molten, e.g., about thirty minutes, just prior to being admixed incrementally with the remaining ingredients of the base such as the plasticizer, fillers, the bulking agent or sweeteners, the softener and coloring agents to plasticize the blend as well as to modulate the hardness, viscoelasticity and formability of the base, and the flavor-enhancing composition (as a concentrate with other additives or separately). Mixing is continued until a uniform mixture of the gum composition is obtained. Thereafter the gum composition mixture may be formed into desirable gum shapes.

One embodiment is method for the manufacture of a comestible, comprising: combining a comestible composition, a medicament for the treatment of a cough or a cold or flu symptom, a physiological cooling agent, and a high intensity sweetener.

Use of the above-described compositions provides a method for enhancing the flavor of a comestible product, wherein the method comprises providing the comestible product comprising a comestible composition, a medicament for the treatment of a cough or a cold or flu symptom, a physiological cooling agent, and a high intensity sweetener, to a consumer, and instructing the consumer to apply the comestible product to the oral cavity of an individual and allow the comestible to dissolve (thereby releasing the above-described flavor-enhancing composition from the comestible into the oral cavity). Providing may be accomplished by a manufacturer, distributor, or other seller that makes the product available to the consumer. Instructing may be by means of packaging, package inserts, advertisements, web sites, and the like. Allowing the comestible to release the composition can be by chewing, or allowing the comestible to dissolve.

A method for enhancing the flavor of a comestible product comprises applying the comestible product comprising a comestible composition, a medicament for the treatment of a cough or a cold or flu symptom, a physiological cooling agent, and a high intensity sweetener, to the oral cavity of an individual; and allowing the comestible to release the above-described flavor-enhancing composition from the comestible into the oral cavity, thereby enhancing the flavor of the comestible product.

A method for the treatment of a cough, or a cold or flu symptom, in a subject in need of such treatment, comprises administering to the subject a flavor-enhanced comestible product comprising a comestible composition, a medicament for the treatment of a cough or a cold or flu symptom, a physiological cooling agent, and a high intensity sweetener. In one embodiment, the comestible is a lozenge or hard candy. In another embodiment, the comestible is gum. Advantageously, use of the flavor-enhancing composition can improve the subject's compliance. In one embodiment, depending on the medicament, the comestible is used to treat coughs, allergies, fevers, pain, inflammation, sore throat, sinus problems, and other maladies. In another embodiment, the comestible is used to treat cough or sore throat.

As the composition is defined as comprising multiple components, it will be understood that each component is chemically distinct, particularly in the instance that a single chemical compound may satisfy the definition of more than one component.

The foregoing and other embodiments are further illustrated by the following examples, which are not intended to limit the effective scope of the claims. All parts and percentages in the examples and throughout the specification and claims are by weight of the final composition unless otherwise specified.

### EXAMPLES

Table 1 illustrates amount ranges for exemplary flavor-enhancing concentrates that can be used to mask bitter or unpleasant off-note flavor components in a wide variety of comestibles. All percentages in Table 1 are by weight of the flavor-enhancing concentrate.

In a specific embodiment, the medicament is dextromethorphan.

TABLE 1

| Ingredient | Range 1 | Range 2 | Range 3 |
|---|---|---|---|
| Sucralose | 0–80 | 1–50 | 5–40 |
| Neotame | 0–20 | 0.01–15 | 0.05–10 |
| Sodium Citrate | 0–60 | 0.1–50 | 1–40 |
| Acidulant | 0–60 | 1–50 | 10–40 |
| Menthol | 0–95 | 1–90 | 5–80 |
| WS-3 | 0–15 | 0.01–10 | 0.1–5 |
| Menthyl glutarate | 0–15 | 0.01–10 | 0.1–5 |
| esters | | | |
| Medicament | 0.001–20 | 0.01–10 | 0.1–1 |
| Sodium chloride | 0–60 | 0.1–50 | 1–40 |
| Inosine | 0–60 | 0.1–50 | 1–40 |
| monophosphate | | | |
| Guanylate | 0–60 | 0.1–50 | 1–40 |
| monophosphate | | | |
| Koji aji kokumi | 0–60 | 0.1–50 | 1–40 |
| potentiator | | | |
| Quercetin | 0–60 | 0.1–50 | 1–40 |
| Flavor | 0–10 | 0.00001–10 | 0.0001–1 |

Table 2 illustrates exemplary ranges of the components of the flavor-enhancing compositions in comestible products. All amounts in Table 2 are percent by weight of the comestible product.

In a specific embodiment, the medicament is dextromethorphan.

TABLE 2

| Ingredient | Range 1 | Range 2 | Range 3 |
|---|---|---|---|
| Sucralose | 0–2 | 0.001–1 | 0.005–0.5 |
| Neotame | 0–2 | 0.0001–1 | 0.001–0.5 |
| Sodium Citrate | 0–3 | 0.01–2 | 0.1–1 |
| Acidulant | 0–3 | 0.01–2 | 0.1–1 |
| Menthol | 0–3 | 0.001–3 | 0.01–1 |
| WS-3 | 0–5 | 0.00001–3 | 0.0001–2 |
| Menthyl | 0–5 | 0.000001–3 | 0.0001–2 |
| glutarate esters | | | |
| Sodium | 0.005–0.5 | 0.01–0.3 | 0.02–0.1 |
| chloride | | | |
| Inosine | 0.005–0.8 | 0.01–0.5 | 0.05–0.3 |
| monophosphate | | | |
| Guanylate | 0.001–0.5 | 0.005–0.3 | 0.05–0.1 |
| monophosphate | | | |
| Koji aji kokumi | 0.001–0.5 | 0.005–0.3 | 0.05–0.1 |
| potentiator | | | |
| Quercetin | 0.005–0.8 | 0.01–0.5 | 0.05–0.3 |
| Flavor | 0–10 | 0.000001–10 | 0.00001–1 |
| Dextro- | 0.0001–2 | 0.00025–1 | 0.01–1 |
| methorphan | | | |

Table 3's Examples A-D provide exemplary compositions for flavor-enhanced throat lozenges.

TABLE 3

| | % by weight | | | |
|---|---|---|---|---|
| Component | A | B | C | D |
| Candy Base (sugar, glucose | 90–99.9 | 90–99.9 | 90–99.9 | 90–99.9 |
| syrup 42DE and water) | | | | |
| WS-3 | 0–5 | 0–5 | | 0–5 |
| WS-23 | 0–5 | | 0–5 | |
| Sodium Chloride | | 0.005–0.5 | | |
| Quercetin | | | 0.005–0.8 | |
| Inosine monophosphate | | | | 0.005–0.8 |
| Guanylate monophosphate | | | | 0.001–0.5 |
| Koji Aji | | | | 0.001–0.5 |
| Sucralose | 0–2 | | | 0–2 |
| Ace-K | | 0–2 | | |
| Neotame | 0–2 | | 0–2 | |
| Flavor | 0.01–10 | 0.01–10 | 0.01–10 | 0.01–10 |
| Color solution | 0.01–1.0 | 0.01–1.0 | 0.01–1.0 | 0.01–1.0 |
| Dextromethorphan | 0.0001–2 | 0.0001–2 | 0.0001–2 | 0.0001–2 |

Throat lozenges are prepared from the formulations in Table 3 by thoroughly mixing the sugar/glucose syrup/water together and heating to 146° C. The batch is placed on a cooling table where the remaining ingredients are added. The batch is then kneaded and molded into the desired final shape for the lozenges.

All cited patents, patent applications, and other references are incorporated herein by reference in their entirety. However, if a term in the present application contradicts or conflicts with a term in the incorporated reference, the term from the present application takes precedence over the conflicting term from the incorporated reference.

As used herein the terms “comprising” (also “comprises,” etc.), “having,” and “including” is inclusive (open-ended) and does not exclude additional, unrecited elements or method steps.

The singular forms “a,” “an,” and “the” include plural referents unless the context clearly dictates otherwise.

The endpoints of all ranges directed to the same characteristic or component are independently combinable, and inclusive of the recited endpoint.

The term “combination” is inclusive of a homogeneous or non-homogeneous blend, mixture, or alloy of the named components into an integrated whole. The term “homogeneous” refers to a uniform blend of the components.

The word “or” means “and/or.”

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A flavor-enhancing composition for a comestible product, comprising:
a medicament for the treatment of a cough, or a cold or flu symptom,
a physiological cooling agent, and
a high intensity sweetener.

2. The composition of claim 1, wherein the medicament is an antihistamine, a decongestant, an antitussive, an anti-inflammatory, a homeopathic agent, an expectorant, an anesthetic, a demulcent, an analgesic, an anticholinergic, a throat-soothing agent, an antibacterial agent, an antiviral agent, or a combination of at least two of the foregoing medicaments.

3. The composition of claim 1, wherein the medicament is a decongestant, an antitussive, an anti-inflammatory, an expectorant, a demulcent, an analgesic, a throat-soothing agent, or a combination of at least two of the foregoing medicaments.

4. The composition of claim 1, wherein the medicament is an antitussive, an expectorant, a demulcent, a throat-soothing agent, or a combination of at least two of the foregoing medicaments.

5. The composition of claim 1, wherein the medicament is an antitussive.

6. The composition of claim 1, wherein the physiological cooling agent is xylitol, erythritol, dextrose, sorbitol, p-menthane, menthone, a menthone ketal, a menthone glycerol ketal, menthol, a natural or synthetic derivative of menthol, (−)-(1R,3R,4S)-3-p-menthanol, (−)-(1R,3R,4S)-8-p-menthen-3-ol, p-menthane-2,3-diol, p-menthane-3,8-diol, menthol glyceryl ether, 6-isopropyl-9-methyl-1,4-dioxaspiro[4,5]decane-2-methanol, 3-(1-menthoxy)ethan-1-ol, 3-(1-menthoxy)propan-1-ol, 3-(1-menthoxy)butan-1-ol, menthoxyalkane diols, 3-(1-menthoxy)propane-1,2-diol, 3-(1-menthoxy)-2-methylpropane-1,2-diol, WS-30, p-menthane-3-carboxylic acid glycerol ester, menthol methyl ether, a menthyl ester of an aliphatic or aromatic monocarboxylic acid, menthyl acetate, menthyl lactate, menthyl 3-hydroxybutyrate, menthyl 4-hydroxypentanoate, menthyl salicylate, menthyl pyrrolidone carboxylate, a monomenthyl ester of an aliphatic dicarboxylic acid, monomenthyl glutarate, monomenthyl succinate, a p-menthane carboxamide, an N-aryl menthane carboxamide, N-ethyl-p-menthane-3-carboxamide (WS-3), 1-menthylacetic acid N-ethylamide, N,N-dimethyl menthyl succinamide, N-tert-butyl-p-menthane-3-carboxamide (WS-14), ethyl 3-(p-menthane-3-carboxamido)acetate, a menthol glycol carbonate, menthol ethyleneglycol carbonate, menthol propyleneglycol carbonate, trimethylcyclohexanol, isopulegol, N-methyl-2-isopropyl-bicyclo(2.2.1)heptane-2-carboxamide, an acyclic carboxamide, N,2,3-trimethyl-2-isopropyl butanamide (WS-23), N-ethyl-trans-2-cis-6-nonadienamide, 1-methyl-cyclohexanecarboxylic acid (3-methoxy-phenyl)-amide, 1-methyl-cyclohexanecarboxylic acid (4-cyano-phenyl)-amide, 2-methyl-bicyclo[2.2.1]hept-5-ene-2-carboxylic acid (4-cyano-pheny)-amide, 2-methyl-bicyclo[2.2.1]hept-5-ene-2-carboxylic acid (4-methoxy-phenyl)-amide, 3-isopropyl-1-methyl-cyclopentanecarboxylic acid (4-methoxy-phenyl)-amide, 3-isopropyl-1-methyl-cyclopentanecarboxylic acid (3-cyano-phenyl)-amide, adamantane-1-carboxylic acid (4-methoxy-phenyl)-amide, 2-tert-butyl-cyclopentanecarboxylic acid (4-methoxy-phenyl)-amide, 2-tert-butyl-cyclohexanecarboxylic acid (2-methoxy-phenyl)-amide, 2-tert-butyl-cyclopentanecarboxylic acid (4-hydroxymethyl-phenyl)-amide, 2-tert-butyl-cyclopentanecarboxylic acid (4-acetyl-phenyl)-amide, 2-tert-butyl-cyclopentanecarboxylic acid (4-cyano-phenyl)-amide, 2-tert-butyl-cyclohexanecarboxylic acid (4-hydroxymethyl-phenyl)-amide, 2-tert-butyl-cyclohexanecarboxylic acid (4-acetyl-phenyl)-amide, and 2-tert-butyl-cyclohexanecarboxylic acid (4-cyano-phenyl)-amide, a thienopyrimidine cooling agent, 2-mercapto-cyclodecanone, Japanese mint oil, peppermint oil, eucalyptus extract, an edible salt of one of the foregoing physiological coolants, or a combination thereof.

7. The composition of claim 1, wherein the physiological cooling agent is menthol, a menthyl ester, a menthyl carboxamide, or a combination of at least two of the foregoing physiological cooling agents.

8. The composition of claim 1, wherein the physiological cooling agent is menthyl carboxamide, N-ethyl-p-menthane carboxamide, monomenthyl succinate, monomenthyl methyl succinate, monomenthyl glutarate, menthyl 2-pyrrolidone-5-carboxylate, monomenthyl 3-methylmaleate, menthyl acetate, menthyl lactate, menthyl salicylate, 2-isopropanyl-5-methylcyclohexanol, 3,1-menthoxypropane 1,2-diol, menthane, menthone, a menthone ketal, a menthone glycerol ketal, a menthyl glutarate ester, N-ethyl-p-menthane-3-carboxamide, or a combination of at least two of the foregoing physiological cooling agents.

9. The composition of claim 1, wherein the physiological cooling agent excludes menthol.

10. The composition of claim 1, wherein the high intensity sweetener is a natural water-soluble sweetener, a water-soluble artificial sweetener, a water-soluble sweetener derived from a naturally occurring water-soluble sweetener, a dipeptide based sweetener, a protein based sweetener, or a combination of at least two of the foregoing high intensity sweeteners.

11. The composition of claim 1, wherein the high intensity sweetener is monellin, steviosides, glycyrrhizin, dihydroflavenol, sorbitol, mannitol, maltitol, monatin, an L-aminodicarboxylic acid aminoalkenoic acid ester amide, a water-soluble saccharin salt, a cyclamate salt, an acesulfame salt, the calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide, the free acid form of saccharin, L-aspartyl-L-phenylalanine methyl ester, L-alpha-aspartyl-N-(2,2,4,4-tetramethyl-3-thietanyl)-D-alaninamide hydrate, the methyl ester of L-aspartyl-L-phenylglycerine, L-aspartyl-L-2,5-dihydrophenyl-glycine, L-aspartyl-2,5-dihydro-L-phenylalanine, L-aspartyl-L-(1-cyclohexen)-alanine, neotame, steviosides, chloro-1′-deoxysucrose, 4-chloro-4-deoxygalactosucrose, 4,1′-dichloro-4,1′-dideoxygalactosucrose, 1′,6′-dichloro 1′,6′-dideoxysucrose, 4,1′,6′-trichloro-4,1′,6′-trideoxygalactosucrose, 4,6,6′-trichloro-4,6,6′-trideoxygalactosucrose, 6,1′,6′-trichloro-6,1′,6′-trideoxysucrose, 4,6,1′,6′-tetrachloro4,6,1′,6′-tetradeoxygalactosucrose, 4,6,1′,6′-tetradeoxysucrose, thaumaoccous danielli, talin, or a combination of at least two of the foregoing high intensity sweeteners.

12. The composition of claim 1, wherein the high intensity sweetener is neotame, sucralose, or a combination of neotame and sucralose.

13. The composition of claim 1, wherein the high intensity sweetener is neotame.

14. The composition of claim 1, wherein the high intensity sweetener is sucralose.

15. The composition of claim 1, wherein a bitter or unpleasant off-note taste is imparted by the medicament or the high intensity sweetener or the physiological cooling agent or a combination of two of the foregoing, and wherein a bitter or unpleasant off-note taste of a combination of the medicament, the high intensity sweetener, and the physiological cooling agent is less than the bitter or unpleasant off-note taste imparted by the medicament or the high intensity sweetener or the physiological cooling agent or the combination of two of the foregoing.

16. The composition of claim 1, wherein the composition further comprises a flavor modulator, a flavor potentiator, a flavorant, an additional sweetener, a coloring agent, an additional medicament, a breath freshener, an antioxidant, an acidulant, a buffering agent, an additional coolant, a mouth moistener, or a combination of at least two of the foregoing additives.

17. The composition of claim 1, wherein the composition further comprises a flavor potentiator selected from the group consisting of sodium chloride, monosodium glutamate, quercetin, adenosine monophosphate, inosine monophosphate, guanylate monophosphate, edible salts of the foregoing, and combinations of at least two of the foregoing.

18. The composition of claim 1, wherein the composition further comprises a flavorant, a coloring agent, an acidulant, a buffering agent, or a combination of at least two of the foregoing additives.

19. The composition of claim 1, wherein the composition further comprises a sweetness potentiator.

20. The composition of claim 1, wherein the composition further comprises a sweetness potentiator, wherein the sweetness potentiator is monoammonium glycyrrhizinate, a licorice glycyrrhizinate, citrus aurantium, alapyridaine, alapyridaine (N-(1-carboxyethyl)-6-(hydroxymethyl)pyridinium-3-ol) inner salt, miraculin, curculin, strogin, mabinlin, gymnemic acid, cynarin, glupyridaine, a pyridinium-betain compound, sugar beet extract, neotame, thaumatin, neohesperidin dihydrochalcone, a hydroxybenzoic acid, 2-hydroxybenzoic acid, 3-hydroxybenzoic acid, 4-hydroxybenzoic acid, 2,3-dihydroxybenzoic acid, 2,4-dihydroxybenzoic acid, 2,5-dihydroxybenzoic acid, 2,6-dihydroxybenzoic acid, 3,4-dihydroxybenzoic acid, 3,5-dihydroxybenzoic acid, 2,3,4-trihydroxybenzoic acid, 2,4,6-trihydroxybenzoic acid, 3,4,5-trihydroxybenzoic acid, 4-hydroxyphenylacetic acid, 2-hydroxyisocaproic acid, 3-hydroxycinnamic acid, 3-aminobenzoic acid, 4-aminobenzoic acid, 4-methoxysalicylic acid, 2-(4-hydroxy-3-methoxyphenyl)-1-(2,4,6-trihydroxyphenyl)ethanone, 1-(2,4-dihydroxyphenyl)-2-(4-hydroxy-3-methoxyphenyl)ethanone, 1-(2-hydroxy-4-methoxyphenyl)-2-(4-hydroxy-3-methoxyphenyl)ethanone, 2,4-dihydroxy-N-[(4-hydroxy-3-methoxyphenyl)methyl]benzamide, 2,4,6-trihydroxy-N-[(4-hydroxy-3-methoxyphenyl)methyl]benzamide, 2-hydroxy-N-[(4-hydroxy-3-methoxyphenyl)methyl]benzamide, 4-hydroxy-N-[(4-hydroxy-3-methoxyphenyl)methyl]benzamide, 2,4-dihydroxy-N-[(4-hydroxy-3-methoxyphenyl)methyl]benzamide, 2,4-dihydroxy-N-[2-(4-hydroxy-3-methoxyphenyl)ethyl]benzamide, N-[(3-ethoxy-4-hydroxyphenyl)methyl]-2,4-dihydroxy-benzamide, N-[(3,4-dihydroxyphenyl)methyl]-2,4-dihydroxy-benzamide, tagatose, trehalose, maltol, ethyl maltol, vanilla extract, vanilla oleoresin, vanillin, sugar beet extract, sugarcane leaf essence, a compound that respond to a G-protein coupled receptor, an edible salt of the foregoing, or a combination of at least two of the foregoing sweetness potentiators.

21. The composition of claim 1, wherein the composition further comprises a sweetness potentiator, wherein the sweetness potentiator is 2-hydroxybenzoic acid, 3-hydroxybenzoic acid, 3,4-dihydroxybenzoic acid, an edible salt of the foregoing, or a combination of at least two of the foregoing sweetness potentiators.

22. The composition of claim 1, wherein
the medicament comprises dextromethorphan,
the physiological cooling agent comprises menthol, and
the high intensity sweetener comprises neotame, sucralose, or a combination thereof.

23. The composition of claim 1, wherein the comestible product is a chewing gum.

24. The composition of claim 1, wherein the comestible product is a confectionery.

25. A flavor-enhancing composition for a comestible product, comprising:
about 1 to about 50 weight percent sucralose;
about 0.01 to about 15 weight percent neotame;
about 0.1 to about 50 weight percent sodium citrate;
about 1 to about 50 weight percent acidulant;
about 1 to about 90 weight percent menthol;
about 0.01 to about 20 weight percent N-ethyl-p-menthane-3-carboxamide, menthyl glutarate, or a combination thereof; and
about 0.01 to about 10 weight percent dextromethorphan;
wherein all weight percents are based on the total weight of the flavor-enhancing composition.

26. The composition of claim 1, wherein the comestible product is a chewing gum.

27. The composition of claim 1, wherein the comestible product is a confectionery.

28. A method for the manufacture of a flavor-enhancing composition for a comestible product, comprising:
combining
a medicament for the treatment of a cough, or a cold or flu symptom,
a physiological cooling agent, and
a high intensity sweetener.

29. The method of claim 28, wherein
the medicament comprises dextromethorphan,
the physiological cooling agent comprises menthol, and
the high intensity sweetener comprises neotame, sucralose, or a combination thereof.

30. A flavor-enhanced comestible product comprising:
a comestible composition,
a medicament for the treatment of a cough, or a cold or flu symptom,
a physiological cooling agent, and
a high intensity sweetener.

31. The flavor-enhanced comestible product of claim 30, wherein
the medicament comprises dextromethorphan,
the physiological cooling agent comprises menthol, and
the high intensity sweetener comprises neotame, sucralose, or a combination thereof.

32. A method for the manufacture of a comestible, comprising:
combining
a comestible composition,
a medicament for the treatment of a cough, or a cold or flu symptom,
a physiological cooling agent, and
a high intensity sweetener.

33. The method of claim 32, wherein
the medicament comprises dextromethorphan,
the physiological cooling agent comprises menthol, and
the high intensity sweetener comprises neotame, sucralose, or a combination thereof.

34. A method for improving patient compliance with medicament dosing by enhancing the flavor of a comestible product, comprising:
providing to a consumer a comestible product comprising
a comestible composition,
a medicament for the treatment of a cough, or a cold or flu symptom,
a physiological cooling agent, and
a high intensity sweetener; and
instructing the consumer to apply the comestible product to the oral cavity of an individual and allow the comestible to dissolve.

35. A method for enhancing the flavor of a comestible product comprising:
adding to a comestible composition
a medicament for the treatment of a cough, or a cold or flu symptom,
a physiological cooling agent, and
a high intensity sweetener.

36. The method of claim 35, wherein
the medicament comprises dextromethorphan,
the physiological cooling agent comprises menthol, and
the high intensity sweetener comprises neotame, sucralose, or a combination thereof.

37. A method for enhancing the flavor of a comestible product comprising:
applying to the oral cavity of an individual a comestible product comprising
a comestible composition,
a medicament for the treatment of a cough, or a cold or flu symptom,
a physiological cooling agent, and
a high intensity sweetener; and
allowing the comestible to release the above-described flavor-enhancing composition from the comestible into the oral cavity, thereby enhancing the flavor of the comestible product.

38. The method of claim 37, wherein
the medicament comprises dextrometholphan,
the physiological cooling agent comprises menthol, and
the high intensity sweetener comprises neotame, sucralose, or a combination thereof.

39. A method for the treatment of a cough, or a cold or flu symptom, in a subject in need of such treatment, the method comprising:
administering to the subject a flavor-enhanced comestible product comprising
a comestible composition,
a medicament for the treatment of a cough, or a cold or flu symptom,
a physiological cooling agent, and
a high intensity sweetener.

40. The method of claim 39, wherein
the medicament comprises dextromethorphan,
the physiological cooling agent comprises menthol, and
the high intensity sweetener comprises neotame, sucralose, or a combination thereof.
